(19) United States
(12) Patent Application Publication
(10) Pub. No.: US 2023/0157803 A1
(43) Pub. Date: May 25, 2023

(54) NON-TAVR LEFT-SIDED HEART PROCEDURE EMBOLIC PARTICLE PROTECTION DEVICE

(71) Applicant: BOSTON SCIENTIFIC SCIMED, INC., Maple Grove, MN (US)

(72) Inventor: Daniel William Pasche, Brooklyn Center, MN (US)

(73) Assignee: BOSTON SCIENTIFIC SCIMED, INC., Maple Grove, MN (US)

(21) Appl. No.: 17/992,028

(22) Filed: Nov. 22, 2022

Related U.S. Application Data
(60) Provisional application No. 63/282,442, filed on Nov. 23, 2021.

(51) Int. Cl. A61F 2/01

(52) U.S. Cl. CPC A61F 2/013; A61F 2/011

(57) ABSTRACT

An embolic protection system may include a delivery catheter, a core wire slidably disposed within a lumen of the delivery catheter, an expandable support frame, and a filter element coupled to the expandable support frame. The filter element is a material configured to allow blood flow therethrough while blocking a passage of embolic debris. The embolic protection device may be positioned within the ascending aorta with a distal end located between the aortic valve and the coronary arteries.

### CROSS-REFERENCE TO RELATED APPLICATIONS

This application claims the benefit of priority of U.S. Provisional Application No. 63/282,442 filed Nov. 23, 2021, the entire disclosure of which is hereby incorporated by reference.

### TECHNICAL FIELD

In general, the present disclosure relates to medical devices for filtering blood, and more particularly, in certain embodiments, to a system of filters for protecting the coronary arteries from emboli, debris and the like dislodged during an endovascular or cardiac procedure, and methods for using such medical devices.

### BACKGROUND

Left-sided heart procedures include transcatheter aortic valve replacement (TAVR), mitral valve annuloplasty, repair or replacement, left atrial appendage closure (LAAC), ablation, etc. During such left-sided heart procedures, embolic debris may be released into the bloodstream from dislodgement of tissue, thrombus, calcification, or even from large particulates from the medical device itself. The clinical impact of this embolic particle release into the bloodstream is ischemia in downstream organs. The most clinically significant downstream organ for left-side heart procedures is the brain, where embolic debris may result in a cerebrovascular accident (CVA), stroke, or transient ischemic attach (TIA). The kidneys may also be damaged by embolic particles, as well as the limbs, heart (myocardial infarction), and many other organs.

TAVR procedures have a significantly higher stroke rate than other left-sided heart procedures, so the need for cerebral embolic protection is becoming well understood. However, other left-sided heart procedures such as mitral valve procedures, LAAC, and ablation may also experience significant stroke rates. Stroke rates for TAVR, mitral valve procedure, LAAC, and ablation procedures all average in the single digits, but studies have indicated that Silent Brain Infarcts (SBIs), which are ischemic areas that can be detected in the brain but do not produce acute neurological effects, are far more common. For example, while strokes occur in about 9% of transcatheter aortic valve implantation (TAVI) patients, SBIs occur in about 74%. While strokes are under-diagnosed, their negative impacts are well understood. SBIs are less well-understood than strokes, and they have long been known to be linked to increased risk of dementia and overall cognitive decline.

There exist devices for protecting cerebral arteries by either collecting (filters) or deflecting (deflectors) debris during TAVR procedures. There is a need for similar protection devices designed for use in non-TAVR procedures. Of the known medical devices, delivery systems, and methods of providing embolic protection, each has certain advantages and disadvantages. There is an ongoing need to provide alternative medical devices and methods as well as alternative methods for manufacturing and using medical devices.

### SUMMARY

This disclosure provides design, material, manufacturing method, and use alternatives for medical devices. An example embolic protection device includes a delivery catheter having a lumen extending therethrough, a core wire slidably disposed within the lumen of the delivery catheter, the core wire having a distal end region, an expandable support frame including a cylindrical distal region and a conical proximal region, the conical proximal region coupled to the distal end region of the core wire, a membrane disposed over the expandable support frame, the membrane comprising a material configured to allow blood flow therethrough while blocking a passage of embolic debris, and a plurality of anchors coupled to the expandable support frame.

Alternatively or additionally to the embodiment above, the cylindrical distal region defines a distal terminal end of the expandable support frame, wherein the distal terminal end has a diameter substantially the same as a diameter of the cylindrical distal region.

Alternatively or additionally to any of the embodiments above, the expandable support frame is self-expanding.

Alternatively or additionally to any of the embodiments above, the expandable support frame includes a plurality of struts, wherein the plurality of anchors is cut from the plurality of struts.

Alternatively or additionally to any of the embodiments above, the plurality of anchors is configured to extend radially away from the plurality of struts when the delivery catheter is withdrawn from the expandable support frame, and to be pushed back against the plurality of struts when the delivery catheter is moved distally to recapture and re-constrain the expandable support frame.

Alternatively or additionally to any of the embodiments above, at least some of the plurality of anchors project through the membrane.

Alternatively or additionally to any of the embodiments above, when expanded, the expandable support frame has an outer diameter of between 20 mm and 50 mm.

Alternatively or additionally to any of the embodiments above, a length of the cylindrical distal region is greater than a length of the conical proximal region.

Alternatively or additionally to any of the embodiments above, a total length of the expandable support frame is between 25 mm and 100 mm.

An example method of protecting coronary arteries from embolic material released during left-sided heart procedures includes moving a catheter through a descending aorta, across an aortic arch, and into an ascending aorta adjacent an aortic valve, delivering an embolic protection device through the catheter, the embolic protection device including a self-expanding support frame and a filter membrane coupled to the self-expanding support frame, and at least one of proximally retracting the catheter and distally advancing the embolic protection device to deploy the self-expanding support frame from the catheter such that a distal end of the self-expanding support frame is secured between the aortic valve and left and right coronary arteries.

Alternatively or additionally to the embodiment above, a distal region of the self-expanding support frame is cylindrical and the distal end of the self-expanding support frame is round, wherein the distal end has a diameter substantially the same as a diameter of the cylindrical distal region, wherein the self-expanding support frame is positioned with the distal end positioned upstream of an ostium of each of the left and right coronary arteries.

Alternatively or additionally to any of the embodiments above, the self-expanding support frame includes a plurality of struts, wherein a plurality of anchors is cut from the plurality of struts.

Alternatively or additionally to any of the embodiments above, the plurality of struts and the plurality of anchors are made of a shape-memory material.

Alternatively or additionally to any of the embodiments above, proximally retracting the catheter from the embolic protection device allows the plurality of anchors to extend radially away from the plurality of struts, wherein after a medical procedure is performed, the embolic protection device is recaptured by pushing the catheter distally over the embolic protection device to move the plurality of anchors back against the plurality of struts to re-constrain the self-expanding support frame.

Alternatively or additionally to any of the embodiments above, at least some of the plurality of anchors project through the filter membrane.

Alternatively or additionally to any of the embodiments above, the self-expanding support frame is defined by a self-expanding wire hoop and the filter membrane is coupled to the wire hoop and is tapered proximally, wherein the wire hoop is positioned upstream of an ostium of each of the left and right coronary arteries and the filter membrane extends proximally within the ascending aorta.

Alternatively or additionally to any of the embodiments above, when fully expanded, an outer diameter of the self-expanding support frame is between 20 mm and 50 mm.

Alternatively or additionally to any of the embodiments above, a distal end of the filter membrane is coupled to a core wire extending through the catheter, wherein delivering the embolic protection device includes delivering the wire hoop to a position between the aortic valve and the ostium of each of the left and right coronary arteries, followed by proximally retracting the catheter over the core wire to allow the self-expanding wire hoop to expand and engage an inner surface of the ascending aorta and deploy the filter membrane over the ostium of each coronary artery and proximally within the ascending aorta.

Alternatively or additionally to any of the embodiments above, a portion of the filter membrane between the wire hoop and the core wire is devoid of any support structure.

Another example method of protecting a patient's coronary arteries includes moving a delivery catheter through the patient's descending aorta, across an aortic arch and down an ascending aorta to a position in which a distal end of the delivery catheter is adjacent the coronary arteries, the delivery catheter containing a core wire slidably disposed within the delivery catheter, the core wire having a distal end coupled to a proximal end of a protection device, the protection device comprising a self-expanding support frame, a filter membrane coupled to the self-expanding support frame, the method further including proximally retracting the delivery catheter to deploy a distal end of the protection device from a distal end of the delivery catheter, positioning the distal end of the self-expanding support frame between the patient's aortic valve and the coronary arteries, and withdrawing the delivery catheter proximally to allow the protection device to fully expand within the ascending aorta.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 illustrates an example embolic protection device;

FIG. 2 illustrates an example anchor member cut from a strut;

FIG. 3. illustrates the embolic protection device of FIG. 1 disposed within the aorta; and

FIG. 4 illustrates another example embolic protection device disposed within the aorta.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified. The term “substantially” when used in reference to two dimensions being “substantially the same” shall generally refer to a difference of less than or equal to 5%.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream” and “downstream” refer to a direction, position or location relative to the direction of blood flow through a particular element or location, such as a vessel (i.e., the aorta), a heart valve (i.e., the aortic valve), and the like.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently — such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

As awareness of the frequency of neurological effects and the severity of the impacts on patient well-being increases, demand for medical device solutions is increasing. Already a number of options for peri-procedural embolic protection exist, but current options are targeted at TAVR procedures and are designed to be placed in the aortic arch. The current options fall into three general categories, each of which have significant drawbacks/limitations. Most current options simply deflect embolic particles away from cerebral arteries, directing the particulates to cause damage elsewhere in the body. Of the remaining options, none offer full protection (even so-called “complete protection devices” sit in the aortic arch, leaving the coronary arteries unprotected). A few “complete protection devices” sit across the diameter of the aortic arch and thus capture most particulates, but their designs leave the coronary arteries unprotected and the devices are often compromised to allow passage of TAVR catheters, increasing the likelihood of embolic particles escaping around the perimeter of the protection device.

While current embolic protection devices are designed for TAVR, demand is developing for options for other left-sided heart procedures. One example is LAAC procedures where there is persistent thrombus present in the LAA, where the risk of thrombus embolization during LAAC device implantation makes embolic protection critical. Preliminary studies have also indicated that embolic protection for left-sided procedures other than TAVR can have a significant positive effect. In some small studies of mitral and ablation procedures, embolic protection devices caught debris in 100% of patients. Stroke rates for LAAC procedures are similar to mitral and ablation procedures, so it is likely reasonable to assume that embolic protection for LAAC would be similarly beneficial. In some cases, users have placed devices designed for use in the LAA in the ascending aorta for embolic protection. This off-label use of the devices represents an unmet need significant enough that some users are developing ad-hoc methods of addressing it.

A device has been designed for placement in the ascending aorta to provide full, optimized, peri-procedural downstream protection from embolic particles released during left-sided heart procedures other than TAVR. “Full” protection means that the device has the capability of protecting the coronary arteries as well as the rest of the body. “Optimized” protection means that the device is intended to span the full diameter of the aorta without intended allowance for another device to pass by, allowing for maximized apposition to the aortic wall and minimized peri-device flow. “Peri-procedural” protection means providing protection just before, during, and for a short period after the procedure. The device may be used to trap and/or deflect particles in other blood vessels within a subject, and can also be used outside of the vasculature. The device described herein is generally adapted to be delivered percutaneously to a target location within a subject, but can be delivered in any suitable way and need not be limited to minimally-invasive procedures.

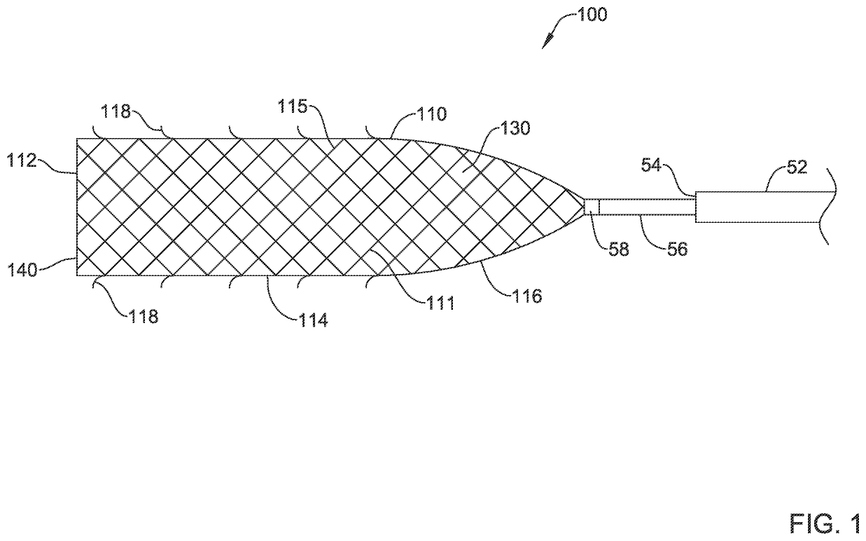

FIG. 1 is a side view of an embodiment of an embolic protection system 100 in a delivered, expanded configuration. The embolic protection system 100 may include an embolic protection device in the form of a filter assembly 110 coupled (e.g., crimped, welded, soldered, etc.) to a flexible elongate core wire 56. The filter assembly 110 may be delivered through a catheter such as delivery catheter 52 having a lumen extending therethrough and an atraumatic distal tip 54. The flexible elongate core wire 56 may be slidingly disposed within the lumen of the delivery catheter 52. The distal end region of the core wire 56 may be coupled to a proximal end of the filter assembly 110. The filter assembly 110 may include an expandable support frame 111 and filter element 130. The filter assembly 110 is configured to be radially compressed within the delivery catheter 52 during delivery. The core wire 56 may have sufficient rigidity to push the attached filter assembly 110 distally out of the delivery catheter 52 to deploy the filter assembly 110, as shown in FIG. 1. In other embodiments, the core wire 56 may be too flexible to push the filter assembly, and deployment occurs when the delivery catheter 52 is withdrawn proximally from the filter assembly 110. The core wire 56 may be proximally withdrawn into the delivery catheter 52 and/or the catheter 52 may be advanced distally over the filter assembly 110 in order to compress the filter assembly 110 for removal from the body after use.

In some embodiments, the support frame 111 may be self-expandable. The support frame 111 may include a distal region 114 and a proximal region 116. In some embodiments, the distal region 114 may be cylindrical, and the proximal region 116 may be conical. It will be understood that the distal region 114 and the proximal region 116 may have other shapes. The proximal end of the proximal region 116 may be coupled to the distal end region of the core wire 56. The support frame 111 may be structured as an expandable stent configured to generally provide expansion support to the filter element 130 when the filter assembly 110 is in the expanded state (as shown in FIG. 1). When fully expanded, the support frame 111 may engage the inner walls of the aorta to secure the filter assembly 110 during use. In the expanded state, the filter element 130 may be configured to filter fluid (e.g., blood) flowing through the filter assembly 110 and to inhibit or prevent particles (e.g., embolic material) from flowing through the filter assembly 110 by capturing the particles in the filter element 130. While a single filter assembly 110 is illustrated in FIG. 1, it will be understood that two or more filter assemblies 110 may be disposed linearly along the core wire 56.

The support frame 111 may be configured to engage or appose the inner walls of a blood vessel (e.g., aorta) in which the filter assembly 110 is expanded such that the filter assembly 110 is sealed against the wall of the aorta to ensure that most, if not all, blood flow exiting the aortic valve flows through the filter element 130. The support frame 111 may be constructed of a super-elastic material with stress-induced martensite due to confinement in the catheter 52. In some embodiments, the support frame 111 may comprise a shape-memory material configured to self-expand upon a temperature change (e.g., heating to body temperature), or a super-elastic material configured to self-expand upon relief of stresses applied to load the support frame 111 into the catheter 52, or a combination thereof. The support frame 111 may be made of, for example, nickel titanium (e.g., nitinol), nickel titanium niobium, chromium cobalt (e.g., MP35N, 35NLT), copper aluminum nickel, iron manganese silicon, silver cadmium, gold cadmium, copper tin, copper zinc, copper zinc silicon, copper zinc aluminum, copper zinc tin, iron platinum, manganese copper, platinum alloys, cobalt nickel aluminum, cobalt nickel gallium, nickel iron gallium, titanium palladium, nickel manganese gallium, stainless steel, combinations thereof, and the like.

The support frame 111 may comprise a wire (e.g., having a round (e.g., circular, elliptical) or polygonal (e.g., square, rectangular) cross-section). For example, in some embodiments, the support frame 111 may include at least one piece of nitinol wire shape set into a woven or braided distal region 114 and proximal region 116. In other embodiments, the support frame 111 may be formed from laser cut nitinol (or other suitable material). In both the woven/braided wire and laser cut embodiments, the support frame 111 includes substantially straight regions between cross-over points (woven/braided) or intersections (laser cut). These straight regions may be considered struts 115, such that the support frame 111 defines a plurality of struts 115.

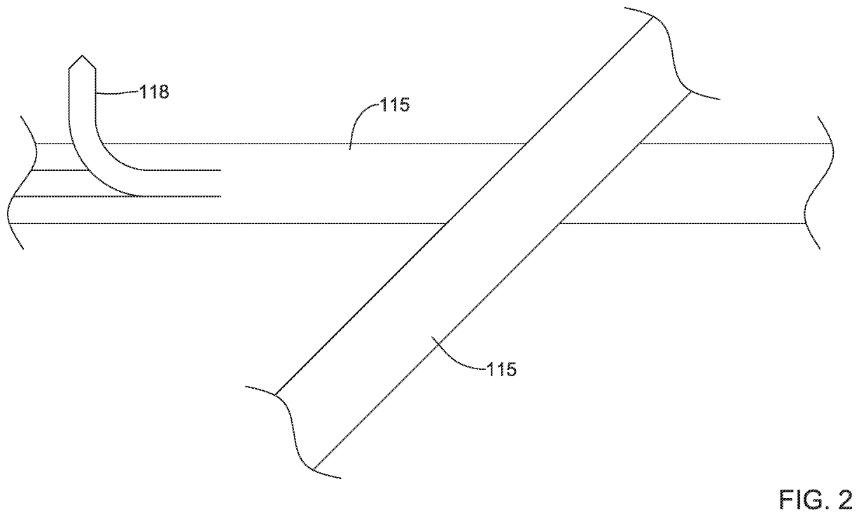

In some embodiments, a plurality of anchors 118 may be coupled to the expandable support frame 111. The anchors 118 may be configured to engage the walls of the aorta to aid in securing the filter assembly 110 to the aorta during use. The plurality of anchors 118 may extend radially outward from the struts 115 when the delivery catheter 52 is withdrawn from the support frame 111 and the support frame 111 expands, as shown in FIG. 1. The plurality of anchors 118 may be configured to be pushed against the plurality of struts 115 when the delivery catheter 52 is moved distally over the filter assembly 110 to recapture and re-constrain the support frame 111. The anchors 118 may be cut from the struts such that the anchors 118 and struts 115 form a monolithic structure, as illustrated in FIG. 2. In other embodiments, the anchors 118 may be formed separately and attached to the support frame 111, such as by welding, soldering, suturing, or adhesive. In some embodiments the plurality of struts 115 and the plurality of anchors 118 are made of a shape-memory material, such as nitinol.

The distal region 114 of the support frame 111 may form a shape of an opening 140 defining the distal terminal end 112 of the filter assembly 110. The opening 140 may be circular, elliptical, or any shape that can appropriately appose sidewalls of a vessel such as the ascending aorta 26, aortic arch 30, etc. The filter assembly 110 may be inserted such that it has a generally distally-facing opening 140. In other embodiments, the opening 140 may be proximally facing. The orientation (e.g., proximal facing or distal facing) of the opening 140 relative to the filter assembly 110 may vary depending on where the access incision is located.

The support frame 111 may include a radiopaque marker such as a coil wrapped around or coupled to the distal terminal end 112 to aid in visualization under fluoroscopy. In some embodiments, the support frame 111 may be formed from or coated with a radiopaque material, such as tantalum, platinum iridium or other suitable material, in order to make the entire support frame 111 visible under fluoroscopy. In some embodiments, a radiopaque marker such as a coil or band 58 may be disposed at the junction between the proximal end of the conical proximal region and the distal end region of the core wire 56.

In some embodiments, the length of the distal region 114 may be greater than the length of the proximal region 116. For example, the total length of the expandable support frame 111 may be between 25 mm and 100 mm, with the distal region 114 making up 60% to 90% of the total length of the support frame 111. In some embodiments, the distal region 114 may be about 66% to 75% of the total length of the support frame 111. In some embodiments, the opening 140 at the distal terminal end 112 may have a diameter substantially the same as the diameter of the distal region 114. This wide distal terminal end 112 of the support frame 111 may engage the wall of the aorta around the entire circumference of the aorta to allow for a large volume of blood to flow through the filter assembly 110 without reducing blood flow. When fully expanded, the expandable support frame 111, and particularly the distal region 114, may have an outer diameter of between 20 mm and 50 mm. In some embodiments, the outer diameter of the fully expanded support frame 111 may be larger than the inner diameter of the treatment site to allow for exertion of slight radial force by the expanding support frame 111 against the aortic wall to keep the device in place while implanted. For example, the outer diameter of the support frame 111 may be about 105% of the inner diameter of the vessel in which it will be implanted, so that when fully expanded, the outer diameter of the distal region 114 of the self-expanding support frame 111 is greater than the inner diameter of the ascending aorta 26 such that proximally retracting the catheter 52 anchors the self-expanding support frame 111 to the inner walls of the ascending aorta 26.

In some embodiments, the filter element 130 may be a filtering membrane 130 disposed over the entire expandable support frame 111, including the distal region 114 and the proximal region 116. In some embodiments, the anchors 118 may project through the membrane 130. The membrane 130 may be formed from a filtering material configured to allow blood flow therethrough while blocking a passage of embolic debris. In this way, blood flows in through the distal terminal end 112 and out through the proximal region 116, but any embolic debris is trapped within the filter assembly 110. The membrane 130 may include pores configured to allow blood to flow through the filter assembly 110, but that are small enough to prevent particles such as embolic material from passing through the filter assembly 110. The membrane 130 may be formed from a polymer (e.g., polyurethane, polytetrafluoroethylene (PTFE)) film mounted to the support frame 111. In some embodiments, the membrane 130 may be made of a nitinol mesh, a stainless-steel mesh, a polymer mesh (e.g., polyether ether ketone (PEEK), or any other suitable material or construction. For example, the membrane 130 may be formed from a knitted or woven material, and the pores may be spaces between the filaments. The membrane 130 may have a thickness between about 0.0001 inches (0.0025 mm) and about 0.03 inches (0.76 mm) (e.g., no more than about 0.0001 inches, about 0.001 inches, about 0.005 inches, about 0.01 inches, about 0.015 inches, about 0.02 inches, about 0.025 inches, about 0.03 inches, ranges between such values, etc.).

The filaments forming the membrane 130 may comprise, for example, polymers, non-polymer materials such as metal, alloys such as nitinol, stainless steel, etc. The pores of the membrane 130 may be circular, elliptical, square, triangular, or other geometric shapes. Certain shapes such as an equilateral triangular, squares, and slots may provide geometric advantage, for example restricting a part larger than an inscribed circle but providing an area for fluid flow nearly twice as large, making the shape more efficient in filtration verses fluid volume. In some embodiments, the membrane 130 may be a sheet structure, and the pores may be laser drilled into or through the membrane 130, although other methods are also possible (e.g., piercing with microneedles, loose braiding or weaving). The pores may have a lateral dimension (e.g., diameter) between about 10 micron (μm) and about 1 mm (e.g., no more than about 10 μm, about 50 μm, about 100 μm, about 150 μm, about 200 μm, about 250 μm, about 300 μm, about 400 μm, about 500 μm, about 750 μm, about 1 mm, and ranges between such values, etc.). Other pore sizes are also possible, for example depending on the desired minimum size of material to be captured.

The material of the membrane 130 may comprise a smooth and/or textured surface that is folded or contracted into the delivery state by tension or compression into a lumen. A reinforcement fabric may be added to or embedded in the membrane 130 to accommodate stresses placed on the membrane 130 during compression. A reinforcement fabric may reduce the stretching that may occur during deployment and/or retraction of the filter assembly 110. The embedded fabric may promote a folding of the filter to facilitate capture of embolic debris and enable recapture of an elastomeric membrane. The reinforcement material may comprise, for example, a polymer and/or metal weave to add localized strength. The reinforcement material may be imbedded into the membrane 130 to reduce thickness. For example, imbedded reinforcement material may comprise a polyester weave mounted to a portion of the membrane 130 near the support frame 111 where tensile forces act upon the support frame 111 and membrane 130 during deployment and retraction of the filter assembly 110 from the catheter 52. In some embodiments, the membrane 130 may be a polyurethane membrane with 100 μm pores.

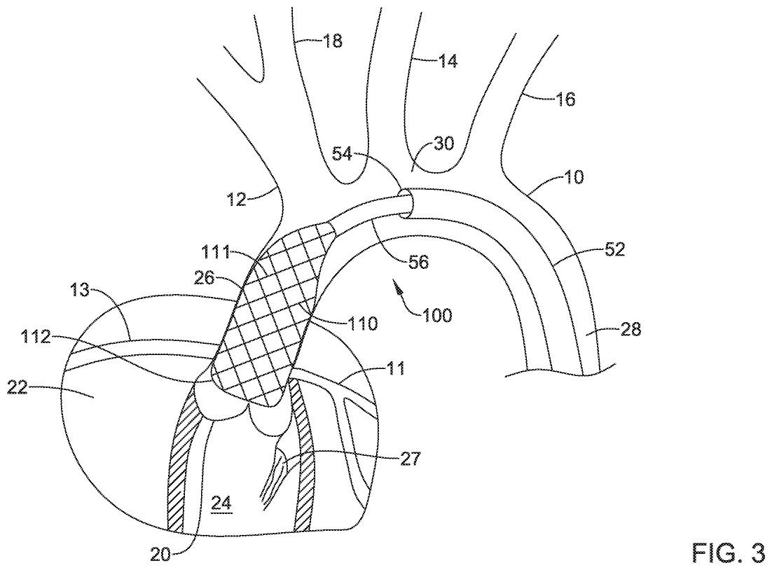

FIG. 3 is a schematic view of a portion of the heart and vasculature with the embolic protection system 100 positioned within the aorta 10. The aorta 10 includes the ascending aorta 26, descending aorta 28, and aortic arch 30. The aorta 10 typically includes three great branch arteries: the brachiocephalic artery or innominate artery 12, the left common carotid artery 14, and the left subclavian artery 16. The innominate artery 12 branches to the right carotid artery 18. The aortic arch 30 is upstream of the left coronary artery 11 and right coronary artery 13. The aortic valve 20 is upstream of the left and right coronary arteries 11, 13. The right ventricle 22, left ventricle 24, and mitral valve 27 of the heart are also shown.

The embolic protection system 100 may be deployed at a target location such as the ascending aorta 26 to capture embolic material or debris from a non-TAVR left-side heart procedure, prior to the debris reaching the coronary arteries 11, 13, the brain, and the rest of the body. In some embodiments, the method of capturing embolic material may include moving the delivery catheter 52 through the descending aorta 28, across the aortic arch 30, and into the ascending aorta 26 adjacent the aortic valve 20, followed by delivering an embolic protection device such as the filter assembly 110, through the catheter 52. In some embodiments, the delivery catheter 52 may comprise a distal curvature, for example based on an intended placement location (e.g., the aortic arch 30).

The embolic protection system 100 may be delivered using a transcatheter placement, for example using femoral access, with the delivery catheter 52 being moved through the vasculature to the descending aorta 28, across the aortic arch 30 and into the ascending aorta 26 adjacent the aortic valve 20, as illustrated in FIG. 3. However, it is contemplated that the embolic protection system 100 may be deployed using a right or left radial access incision, or other location, as desired, in order to access the ascending aorta 26.

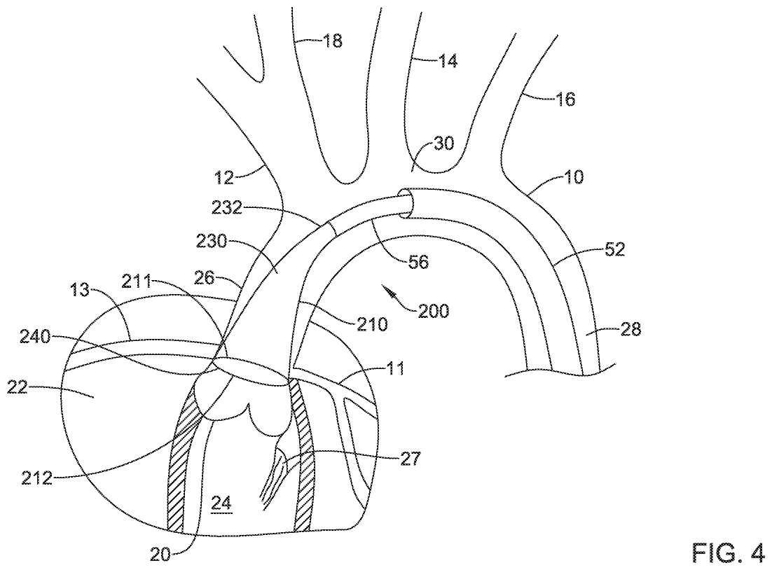

FIG. 4 illustrates another embodiment of an embolic protection system 200 including an embolic protection device in the form of a filter assembly 210 attached to the core wire 56 and deployed through the delivery catheter 52 in the ascending aorta 26. The filter assembly 210 may include a support element or frame 211 and a filter element 230 coupled to the frame 211. The frame 211 may generally provide expansion support to the filter element 230 in the expanded state. In the expanded state, the filter element 230 is configured to filter fluid (e.g., blood) flowing through the filter element 230 and to prevent particles (e.g., embolic material) from flowing through the filter element 230 by capturing the particles in the filter element 230.

The frame 211 may be configured to engage or appose the inner walls of a lumen (e.g., blood vessel such as the ascending aorta 26) in which the frame 211 is expanded. The frame 211 may be self-expandable from a collapsed configuration during delivery within the catheter 52, to an expanded configuration in which it engages the inner walls of the blood vessel to secure the filter assembly 210 within the blood vessel. In some embodiments, the frame 211 comprises a single straight piece of nitinol wire shape set into a circular or elliptical loop or hoop that forms the distal terminal end 212 and opening 240 of the filter assembly 210. The frame 211 may include a radiopaque marker such as a small coil wrapped around or coupled to the hoop to aid in visualization under fluoroscopy. In some embodiments, the self-expandable hoop 211 may be the only frame structure present, with the portion of the filter element 230 between the hoop 211 and the core wire 56 devoid of any support structure. In other embodiments, one or two straight wires may form legs running at an angle coupling the frame 211 to the core wire 56. The filter assembly 210 may have a generally distally-facing opening 240.

The distal terminal end 212 of the frame 211 may be sized to engage the inner wall of the aorta around the entire circumference of the aorta to allow for a large volume of blood to flow through the filter assembly 210 without reducing blood flow. When fully expanded, the frame 211 may have an outer diameter of between 20 mm and 50 mm. In some embodiments, the outer diameter of the fully expanded frame 211 may be larger than the inner diameter of the treatment site to allow for exertion of slight radial force by the expanding frame 211 against the aortic wall to keep the device in place while implanted. For example, the outer diameter of the frame 211 may be about 105% of the inner diameter of the vessel in which it will be implanted, so that when fully expanded, the outer diameter of the self-expanding frame 211 is greater than the inner diameter of the ascending aorta 26 such that proximally retracting the catheter 52 anchors the self-expanding frame 211 to the inner walls of the ascending aorta 26.

The filter element 230 may be a membrane with a distal end disposed over and secured to the frame 211. The filter element 230 extends proximally from the frame 211 and may be tapered to a proximal end 232 that is secured to the core wire 56, such as with adhesive or a crimping element. The frame 211 and filter element 230 together may form a truncated cone with sides of equal length. In other embodiments, the frame 211 and the filter element 230 may form an oblique truncated cone having a nonuniform or unequal length around and along the length of the filter assembly 210. In either shape, the filter assembly 210 may have a shape resembling a windsock, with a distal opening 240 having a first diameter, and tapering down to a second diameter at the proximal end coupled with the core wire 56, where the first diameter is larger than the second diameter.

The filter element 230 may be a filtering membrane 230 formed from a filtering material configured to allow blood flow therethrough while blocking a passage of embolic debris. In this way, blood flows in through the distal terminal end 212 and out through the filter element 230, but any embolic debris is trapped within the filter element 230. The membrane 230 may include pores configured to allow blood to flow through the filter assembly 210, but that are small enough to prevent particles such as embolic material from passing through the filter assembly 210. The membrane 230 may be formed from a polymer (e.g., polyurethane, polytetrafluoroethylene (PTFE)) film mounted to the support frame 211. In some embodiments, the membrane 230 may be made of a nitinol mesh, a stainless-steel mesh, a polymer mesh (e.g., polyether ether ketone (PEEK), or any other suitable material or construction. For example, the membrane 230 may be formed from a knitted or woven material, and the pores may be spaces between the filaments. The membrane 230 may have a thickness between about 0.0001 inches (0.0025 mm) and about 0.03 inches (0.76 mm) (e.g., no more than about 0.0001 inches, about 0.001 inches, about 0.005 inches, about 0.01 inches, about 0.015 inches, about 0.02 inches, about 0.025 inches, about 0.03 inches, ranges between such values, etc.).

The filaments forming the membrane 230 may comprise, for example, polymers, non-polymer materials such as metal, alloys such as nitinol, stainless steel, etc. The pores of the membrane 230 may be circular, elliptical, square, triangular, or other geometric shapes. Certain shapes such as an equilateral triangular, squares, and slots may provide geometric advantage, for example restricting a part larger than an inscribed circle but providing an area for fluid flow nearly twice as large, making the shape more efficient in filtration verses fluid volume. In some embodiments, the membrane 230 may be a sheet structure, and the pores may be laser drilled into or through the membrane 230, although other methods are also possible (e.g., piercing with microneedles, loose braiding or weaving). The pores may have a lateral dimension (e.g., diameter) between about 10 micron (μm) and about 1 mm (e.g., no more than about 10 μm, about 50 μm, about 100 μm, about 150 μm, about 200 μm, about 250 μm, about 300 μm, about 400 μm, about 500 μm, about 750 μm, about 1 mm, and ranges between such values, etc.). Other pore sizes are also possible, for example depending on the desired minimum size of material to be captured.

The material of the membrane 230 may comprise a smooth and/or textured surface that is folded or contracted into the delivery state by tension or compression into a lumen. A reinforcement fabric may be added to or embedded in the membrane 230 to accommodate stresses placed on the membrane 230 during compression. A reinforcement fabric may reduce the stretching that may occur during deployment and/or retraction of the filter assembly 210. The embedded fabric may promote a folding of the filter to facilitate capture of embolic debris and enable recapture of an elastomeric membrane. The reinforcement material may comprise, for example, a polymer and/or metal weave to add localized strength. The reinforcement material may be imbedded into the membrane 230 to reduce thickness. For example, imbedded reinforcement material may comprise a polyester weave mounted to a portion of the membrane 230 near the support frame 211 where tensile forces act upon the support frame 211 and membrane 230 during deployment and retraction of the filter assembly 210 from the catheter 52. In some embodiments, the membrane 230 may be a polyurethane membrane with 100 μm pores.

A method of capturing embolic material from a non-TAVR left-sided heart procedure may include deploying the filter assembly 210 within the ascending aorta 26 prior to performing the heart procedure. The method may include moving the catheter 52 through the descending aorta, across the aortic arch, and into the ascending aorta adjacent the aortic valve. In some embodiments, the catheter 52 may be inserted through the femoral artery. The method also includes delivering a protection device in the form of the filter assembly 210 through the catheter 52, followed by at least one of proximally retracting the catheter 52 and distally advancing the core wire 56 and filter assembly 210 to deploy the self-expanding support frame 211 from the catheter 52 within the ascending aorta 26 such that the distal terminal end 212 of the frame 211 engages the inner walls of the ascending aorta 26 to secure the frame 211 between the aortic valve 20 and the left and right coronary arteries 11, 13, as shown in FIG. 4. This deployment results in the distal terminal end 212 being secured upstream of the ostium of each of the left and right coronary arteries 11, 13, against movement caused by the pressure of blood flowing through the aortic valve 20 and through the filter assembly 210. This placement positions the opening 240 of the filter assembly 210 upstream of the coronary arteries 11, 13, which protects the coronary arteries as well as the rest of the body from emboli or other debris released during the left-side heart procedure.

Once the filter assembly 210 has been deployed within the ascending aorta 26, the non-TAVR left-side heart procedure, such as mitral valve annuloplasty, repair or replacement, left atrial appendage closure (LAAC), ablation, etc., may be performed. Following the completion of the procedure, the filter assembly 210 may be re-sheathed by pushing the catheter 52 distally over the filter assembly 210 to re-constrain the self-expanding support frame 211 within the catheter 52. With the filter assembly 210 and any captured debris positioned within the catheter 52, the entire embolic protection system 200 may then be removed from the body.

The position of the filter assembly 210 in the ascending aorta 26 with the distal terminal end 212 positioned between the aortic valve 20 and the coronary arteries 11, 13 allows the filter assembly 210 to cover the ostium of each of the left and right coronary arteries 11, 13, preventing embolic debris from entering the coronary arteries. This filter assembly 210 and its placement provides an advantage over other conventional embolic protection devices designed for TAVR procedures in which the filters are placed within the innominate artery 12, the left common carotid artery 14, the left subclavian artery 16 and/or a portion of the aortic arch 30. In the TAVR protection devices and procedures, the coronary arteries are not protected and the entire aorta may not be blocked in order to allow for instruments to be passed upstream through the aortic arch into the heart.

In addition to providing the advantage of preventing embolic debris from entering the left and right coronary arteries 11, 13, the frame 211 of the filter assembly 210 is sized to engage the entire diameter of the ascending aorta 26 so no debris may pass into any other vessels, thus protecting the entire body from embolic debris. This is possible because in non-TAVR left-side heart procedures, such as mitral valve annuloplasty, repair or replacement, left atrial appendage closure (LAAC), ablation, etc., the instruments are often delivered to the heart through venous access into the right atrium, and across the intra-atrial septum to the left atrium. The filter assembly 110 extending across the entire width of the ascending aorta 26 will not interfere with the medical procedure.

While the methods and devices described herein may be susceptible to various modifications and alternative forms, specific examples thereof have been shown in the drawings and are described in detail herein. It should be understood, however, that the inventive subject matter is not to be limited to the particular forms or methods disclosed, but, to the contrary, covers all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. In any methods disclosed herein, the acts or operations can be performed in any suitable order and are not necessarily limited to any particular disclosed sequence and not be performed in the order recited. Various operations can be described as multiple discrete operations in turn, in a manner that can be helpful in understanding certain embodiments; however, the order of description should not be construed to imply that these operations are order dependent unless specifically stated as such. Additionally, the structures described herein can be embodied as integrated components or as separate components. For purposes of comparing various embodiments, certain aspects and advantages of these embodiments are described. Not necessarily all such aspects or advantages are achieved by any particular embodiment. Thus, for example, embodiments can be carried out in a manner that achieves or optimizes one advantage or group of advantages without necessarily achieving other advantages or groups of advantages. The methods disclosed herein may include certain actions taken by a practitioner; however, the methods can also include any third-party instruction of those actions, either expressly or by implication.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An embolic protection device comprising:
a delivery catheter having a lumen extending therethrough;
a core wire slidably disposed within the lumen of the delivery catheter, the core wire having a distal end region;
an expandable support frame including a cylindrical distal region and a conical proximal region, the conical proximal region coupled to the distal end region of the core wire;
a membrane disposed over the expandable support frame, the membrane comprising a material configured to allow blood flow therethrough while blocking a passage of embolic debris; and
a plurality of anchors coupled to the expandable support frame.

2. The embolic protection device of claim 1, wherein the cylindrical distal region defines a distal terminal end of the expandable support frame, wherein the distal terminal end has a diameter substantially the same as a diameter of the cylindrical distal region.

3. The embolic protection device of claim 2, wherein the expandable support frame is self-expanding.

4. The embolic protection device of claim 1, wherein the expandable support frame includes a plurality of struts, wherein the plurality of anchors is cut from the plurality of struts.

5. The embolic protection device of claim 4, wherein the plurality of anchors is configured to extend radially away from the plurality of struts when the delivery catheter is withdrawn from the expandable support frame, and to be pushed back against the plurality of struts when the delivery catheter is moved distally to recapture and re-constrain the expandable support frame.

6. The embolic protection device of claim 1, wherein at least some of the plurality of anchors project through the membrane.

7. The embolic protection device of claim 1, wherein when expanded, the expandable support frame has an outer diameter of between 20 mm and 50 mm.

8. The embolic protection device of claim 1, wherein a length of the cylindrical distal region is greater than a length of the conical proximal region.

9. The embolic protection device of claim 8, wherein a total length of the expandable support frame is between 25 mm and 100 mm.

10. A method of protecting coronary arteries from embolic material released during left-sided heart procedures, the method comprising:
moving a catheter through a descending aorta, across an aortic arch, and into an ascending aorta adjacent an aortic valve;
delivering an embolic protection device through the catheter, the embolic protection device including a self-expanding support frame and a filter membrane coupled to the self-expanding support frame; and
at least one of proximally retracting the catheter and distally advancing the embolic protection device to deploy the self-expanding support frame from the catheter such that a distal end of the self-expanding support frame is secured between the aortic valve and left and right coronary arteries.

11. The method of claim 10, wherein a distal region of the self-expanding support frame is cylindrical and the distal end of the self-expanding support frame is round, wherein the distal end has a diameter substantially the same as a diameter of the cylindrical distal region, wherein the self-expanding support frame is positioned with the distal end positioned upstream of an ostium of each of the left and right coronary arteries.

12. The method of claim 11, wherein the self-expanding support frame includes a plurality of struts, wherein a plurality of anchors is cut from the plurality of struts.

13. The method of claim 12, wherein the plurality of struts and the plurality of anchors are made of a shape-memory material.

14. The method of claim 12, wherein proximally retracting the catheter from the embolic protection device allows the plurality of anchors to extend radially away from the plurality of struts, wherein after a medical procedure is performed, the embolic protection device is recaptured by pushing the catheter distally over the embolic protection device to move the plurality of anchors back against the plurality of struts to re-constrain the self-expanding support frame.

15. The method of claim 12, wherein at least some of the plurality of anchors project through the filter membrane.

16. The method of claim 10, wherein the self-expanding support frame is defined by a self-expanding wire hoop and the filter membrane is coupled to the wire hoop and is tapered proximally, wherein the wire hoop is positioned upstream of an ostium of each of the left and right coronary arteries and the filter membrane extends proximally within the ascending aorta.

17. The method of claim 16, wherein when fully expanded, an outer diameter of the self-expanding support frame is between 20 mm and 50 mm.

18. The method of claim 16, wherein a distal end of the filter membrane is coupled to a core wire extending through the catheter, wherein delivering the embolic protection device includes delivering the wire hoop to a position between the aortic valve and the ostium of each of the left and right coronary arteries, followed by proximally retracting the catheter over the core wire to allow the self-expanding wire hoop to expand and engage an inner surface of the ascending aorta and deploy the filter membrane over the ostium of each coronary artery and proximally within the ascending aorta.

19. The method of claim 18, wherein a portion of the filter membrane between the wire hoop and the core wire is devoid of any support structure.

20. A method of protecting a patient's coronary arteries, the method comprising:
moving a delivery catheter through the patient's descending aorta, across an aortic arch and down an ascending aorta to a position in which a distal end of the delivery catheter is adjacent the coronary arteries, the delivery catheter containing a core wire slidably disposed within the delivery catheter, the core wire having a distal end coupled to a proximal end of a protection device, the protection device comprising:
a self-expanding support frame;
a filter membrane coupled to the self-expanding support frame;
proximally retracting the delivery catheter to deploy a distal end of the protection device from a distal end of the delivery catheter;
positioning the distal end of the self-expanding support frame between the patient's aortic valve and the coronary arteries; and
withdrawing the delivery catheter proximally to allow the protection device to fully expand within the ascending aorta.
